(19) United States
(12) Patent Application Publication
(10) Pub. No.: US 2003/0082723 A1
(43) Pub. Date: May 1, 2003

(54) Use of mutated recognition sequences for multiple consecutive recombinase-mediated recombinations in a genetic system

(76) Inventors: Markus Altmann, Munchen (DE); Bernhard Neuhierl, Munchen (DE); Wolfgang Hammerschmidt, Munchen (DE)

Correspondence Address: JENKINS & WILSON, PA, 3100 TOWER BLVD, SUITE 1400, DURHAM, NC, 27707, US

(21) Appl. No.: 10/214,722

(22) Filed: Aug. 7, 2002

(30) Foreign Application Priority Data
Aug. 16, 2001 (DE) 101 40 030.6

(51) Int. Cl. C12P 21/02; C12N 1/18; C12N 15/74

(52) U.S. Cl. 435/69.1; 435/320.1; 435/254.21; 435/254.2; 435/483

(57) ABSTRACT

The present invention relates to an improved method of recombination for site-specific recombinase-mediated recombination using mutated recognition sequences. For this purpose a non-identical pair of recognition sequence mutants is used. Each of the recognition sequence mutants consists of two recognition sequences separated by a spacer. A mutation is introduced into one of the recognition sequences to create, after recombination by a sequence-specific recombinase, a recognition sequence mutant which is no longer recognized by the recombinase.

The present invention relates to an improved method of recombination for site-specific recombination using mutated recognition sequences.

Site-specific recombination is an attractive tool for the manipulation of genetic systems. Unfortunately, the number of possible recombination reactions within a single cell or a genetic system is limited because each recombinase can only be used once and the number of known site-specific recombinases is limited.

One of these is for example the recombinase Cre of E. coli bacteriophage P1, which mediates the site-specific recombination between two identical loxP motifs in an intramolecular or intermolecular manner. Cre recombinase of E. coli bacteriophage P1 is a site-specific recombinase mediating DNA rearrangement via its DNA target sequence, loxP (1). The loxP sequences consist of an 8 bp spacer region flanked by two 13 bp inverted repeats serving as the recognition sequences for DNA binding of Cre (2, 3). The recombination event depends only on these two components and is carried out with absolute reliability. It has been found that similar to the Flp-FRT system of S. cerevisiae the Cre-loxP system effectively catalyzes recombination events in both prokaryotic and eukaryotic cells including those from yeast, plants, insects and mammals. Site-specific recombination events are widely used as tools for conditional genetic alterations in single cells and animals (for a more recent review see (4, 5) and the references cited therein).

A plurality of other site-specific recombination systems exists which are based on a two-component system. It is common to all those systems that they comprise specific repetitive DNA sequences. These sequences in each case consist of two recognition sequences separated by a spacer wherein the recognition sequences are inversely repetitive to each other. In this respect the two components are identical. Besides the examples mentioned above there are also known the Zygosaccharomyces rouxii pSR1, the resolvase-rfsF and the phage Mu Gin recombinase system.

The recombinase systems such as for example the Cre-loxP system may be used for excision, inversion or insertion of DNA segments flanked by recognition sequences because the recombinase mediates intramolecular (excision or inversion) as well as intermolecular (insertion) recombination events. During an excision the region of a DNA sequence between two recognition sequences is excised. Similarly, it is possible to insert circular DNA containing e.g. a loxP sequence into a genetic locus which also contains a loxP sequence. It should be noted, however, that in all of these cases not only the desired reaction occurs but that it is always accompanied by the back reaction.

The properties of the recombination systems, e.g. the Cre system, have been combined with various conventional gene targeting and replacement strategies (4, 5). Generally, conventional genomic alterations are based on a targeted integration of a modified allele. In eukaryotic and prokaryotic cells the integration event is achieved by homologous recombination with regions flanking the allele of interest. A positive genetic marker for the selection of homologous recombination events is obligatory which occur at a low frequency in most of the genetic systems. Therefore it is often desirable to remove this marker in a subsequent step, preferably in association with a remaining wild-type allele. For the removal of the marker gene (or of DNA segments to be deleted) loxP sequences which have been introduced enable efficient excision of the loxP flanked DNA segment in a strictly Cre-dependent manner. The excised fragment is circularized and is lost by degradation while, however, a single loxP sequence remains in the modified gene locus. In a later step, this loxP sequence together with a second loxP sequence may serve as a further site for Cre recombinase whereby undesired recombination events may occur.

The genetic manipulation of E. coli plasmids carrying inserts of more than 100 kbp is a rather novel approach which has been brought about by the cloning of large chromosomal fragments into single copy E. coli plasmids based on F factor or on the bacteriophage P1 replicons called BAC and PAC plasmids, respectively (6-8). The synthetic construction of E. coli plasmids of this size has also been possible (9, 10) as well as the molecular cloning of the complete genome of herpes virus having a size of more than 250 kbps and encoding more than 200 different genes (for a more recent review see ref. 11). Genetic manipulation of BACs and PACs is generally achieved using different homologous recombination protocols in E. coli which requires the use of a selectable marker gene (11, 12). Multiple independent alterations in a single plasmid necessitate the immediate removal of the selectable marker or the subsequent use of different marker genes. About half a dozen different antibiotic resistance genes (and an even larger number of auxotrophic markers) are available but their removal can only be achieved by a relatively small number of site-specific recombination systems of which the combinations Cre-loxP, Flp-FRT, resolvase-rfsF have been most extensively studied. As a consequence, the number of independent alterations within a single DNA molecule is relatively limited.

To increase the repertoire of genetic manipulations using recombinase systems within a single cell there have been for example generated mutations of the Cre-loxP system within the spacer region (13) or the inverted repeats (14) modifying the properties of loxP sequences. A loxP sequence having a modified spacer region can only recombine with equivalent or paired loxP sequences but is unable to undergo Cre-mediated recombination with a wild-type loxP locus or another loxP variation (15). Following Cre-mediated recombination the resulting loxP sequence can still be recognized by Cre. As a consequence, loxP sequences having modified spacer regions cannot be used for consecutive recombination events within the same genetic system.

It has been reported that loxP variations with altered inverted repeat regions promote the stable integration of a loxP-flanked DNA segment into an individual preexisting loxP locus within a plant chromosome (14). The recombination event results in the generation of a mutated loxP sequence carrying modification in both inverted repeats and a second loxP site which is wild-type (14). It has been assumed that the mutated loxP sequence is a poor substrate for Cre recombinase (14) but it has been reported that the system as a whole is pervious and unstable (16), i.e. the loxP mutant has been reported to be still recognized by Cre recombinase as a target sequence.

Therefore the object of the present invention is to create an improved method of recombination to enable multiple and targeted mutations within a genetic system in the course of multiple consecutive homologous and recombinase-mediated recombination events. This object has been achieved by the features set forth in the independent claims. Preferred embodiments and modifications of the invention are presented in the dependent claims.

The problem mentioned above has been solved by the method of recombination according to the invention which utilizes a non-identical pair of recognition sequence mutants. Each of the recognition sequence mutants consists of two recognition sequences separated by a spacer. Mutations have been introduced into one of the recognition sequences while the other corresponds to wild-type. Following recombination by a sequence-specific recombinase a recognition sequence mutant is generated which carries mutations in both of the recognition sequences and thus is no longer recognized by the recombinase. Thus, it cannot be used for further recombinase-mediated recombination. Due to this fact, the back reaction which normally takes place at an equilibrium with the direct reaction (see above) is abolished. Therefore, the reaction is unidirectional. Furthermore, the recognition sequence mutants provided in the beginning (each having only one mutated recognition sequence) may be used several times within the same genetic system because no sequence capable of competing with other recognition sequences introduced (either mutated sequences or wild-type sequences) will be present after the recombination has occurred.

This creates the possibility of introducing a DNA fragment into a genetic system with a higher efficiency as achieved heretofore or recombining an infinite number of segments within a genetic system.

According to the present invention two non-identical recognition sequence mutants are used each carrying mutations in one of the recognition sequences which are inversely repetitive to each other while the respective other recognition sequence corresponds to the wild-type recognition sequence for effecting two or more consecutive recombination events by means of a recombinase within a single genetic system.

The term “recognition sequence mutant” as used in the present context comprises mutations occurring within the wild-type sequences of the following type: point mutations of one nucleotide or a few neighboring nucleotides, mutations affecting several nucleotides, deletions, additions, and nucleotide exchange.

According to a preferred embodiment the two non-identical recognition sequences are loxP mutants, i.e. the two loxP mutants lox 66 and lox 71 corresponding to SEQ. ID. NOS. 1 and 6. Other loxP recognition sequence mutants are SEQ. ID. NOS. 2-5 and 7-10.

More particularly, the method of recombination according to the present invention for carrying out multiple recombinations by means of a recombinase within a single genetic system comprises the following steps:

First, two non-identical recognition sequence mutants are provided in the genetic system. “Genetic system” herein means for example a prokaryotic or eukaryotic cell or also an animal or plant organism. Examples of prokaryotic systems are E. coli, Salmonella species, Bacillus species, bacteriophages. Eukaryotic systems are for example human and animal cells and cell lines of somatic origin, mouse, zebra fish, Drosophila, S. cerevisiae, Xenopus laevis.

The two non-identical recognition sequences each have mutations in one of the recognition sequences (referred to as “inverted repeat” sequences in the wild-type) which are inversely repetitive to each other. In other words, the one mutated recognition sequence of a recognition sequence mutant is inversely repetitive to the mutated recognition sequence of the other recognition sequence mutant. Inversely repetitive sequences generally refers to DNA and RNA sequence elements which are directly or not directly adjacent to each other and have an inverted complementary or almost complementary sequence. As a result, the sequences form so-called inverted repeat sequences.

The other sequence contained in the mutants corresponds to the wild-type sequence. For example, in both loxP mutants the respective other recognition sequence corresponds to the non-mutated loxP wild-type. The two loxP mutants must be aligned to have their wild-type sequences on the side facing the site of recombination. In the course of the recombination event with the corresponding DNA sequence, these sequences will be excised resulting in a loxP mutant consisting of two mutated recognition sequences. According to the invention, this mutant then is no longer subject to recognition by Cre recombinase and thus no longer involved in other Cre-mediated recombination events.

The next step of the method of recombination according to the present invention involves the induction of a sequence-specific recombinase to perform a recombination event leaving—as mentioned above—a recognition sequence mutant with a modified nucleic acid sequence wherein this recognition sequence mutant is no longer recognized by the recombinase. This method may be repeated as often as desired to perform further recombination events.

According to a preferred embodiment the two loxP mutants lox 66 and lox 71 are utilized in the recombination method according to the invention. The orientation of these and also of all other recognition sequence mutants according to the invention is unequivocally determined by the spacer sequence localized between the two recognition sequences (direct or inverted). Usually, a recombination event can only occur if the two recognition sequence mutants are present in a direct orientation to each other. It is important that the respective wild-type sequences are localized on the side which faces the site of recombination, i.e. for example the loxP mutants must be arranged in the following order: mutated lox 66 sequence→wild-type lox 66 sequence→wild-type lox 71 sequence→mutated lox 71 sequence (or vice versa). The nucleic acid sequences disclosed herein are always arranged in 5!→3′ direction.

According to one embodiment, in the recombination method according to the present invention the recognition sequence mutants of SEQ. ID. NOS. 1-5 are flanked in 5′→3′ direction by the sequences ATTCC and TCTCG, and the recognition sequence mutants of SEQ. ID. NOS. 6-10 are flanked in 5′→3′ direction by the sequences GCTTC and CTCTT.

Cre recombinase may be for example generated by expression in a genetic system such as by means of a vector encoding Cre recombinase.

As already mentioned above the genetic system of the present invention may be a prokaryotic or eukaryotic cell, such as a bacterial, yeast, plant, insect or mammalian cell. Similarly, the genetic system may consist of a defined isolated nucleic acid unit for example a plasmid.

The recombination event taking place in the course of the method of recombination according to the invention may comprise an insertion or excision of a DNA sequence.

For example in the course of an excision a DNA sequence may be excised which contains a marker gene. Preferably, these marker genes may be antibiotic resistance genes which for example confer resistance to chloramphenicol, tetracyclin or ampicillin.

For insertion of a DNA segment present on a mobile genetic element such as an extrachromosomal plasmid the starting situation may be for example the following: The DNA segment may be flanked on the left by a lox 66 and on the right by a lox 71 recognition sequence. The orientation of the two lox variants is in the same direction. The DNA segment to be inserted additionally contains a suitable marker gene located adjacent to the gene or genetic element of interest. Preferably, also a single lox 66 recognition sequence is already present on the chromosome of a cell and serves as a target sequence.

After expression of Cre the following structure from right to left will be formed: lockP—inserted DNA segment—lox 66. In this manner, the back reaction, i.e. excision of the inserted DNA segment is impossible due to the blocked lockP recognition sequence. The lox 66 recognition sequence which is still present may be used for further insertions.

The method of recombination according to the present invention is most reliable if during further recombination events the loxP mutant generated in the first recombination event has the same orientation as the loxP mutants provided for carrying out further recombination events, i.e. is in a direct orientation (orientation in the same direction) relative to the spacers. With respect to the term orientation the above explanations regarding lox 66 and lox 71 apply in an analogous fashion.

In the following the present invention will be explained in more detail by means of Examples as well as the accompanying Figures.

### THE FIGURES SHOW:

FIG. 1:

Targeted nucleotide sequences of wild-type (loxP) and mutated (lox66/lox71) loxP sequences. Flanking regions, inverted repeats and the spacer region are separated by blanks. The numbers indicate the positions of the nucleotides within the inverted repeats. Mutated nucleotides are represented by smaller letters.

FIG. 2:

Frequency of Cre-mediated recombination events. Cre recombinase was transiently expressed over night at 30° C. in E. coli which either harbored plasmid p2724 or p2725. The cells were harvested and the plasmids were isolated by standard procedures. Ten pg each of the plasmid preparations were transformed into E. coli plated onto LB agar plates containing ampicillin. The colonies were replica-plated onto LB plates which contained either ampicillin (Amp), chloramphenicol (Cm), or tetracycline (Tc). Growth on this combination of three antibiotics indicated that no recombination had occurred. Resistance to ampicillin and chloramphenicol but sensitivity to tetracycline corresponds to a Cre-mediated recombination between lox66 and lox71 but not with lockP. Any other phenotype indicates undesired recombination events. The frequency of phenotypes of the replica-plated colonies in the presence of different antibiotics is shown. A total of 208 colonies has been evaluated for each test plasmid.

### EXAMPLES

The following test systems show that particular mutations within the inverted repeats of loxP result in mutated loxP sequences which recombine efficiently but form a refractory loxP site after a single round of Cre-mediated recombination.

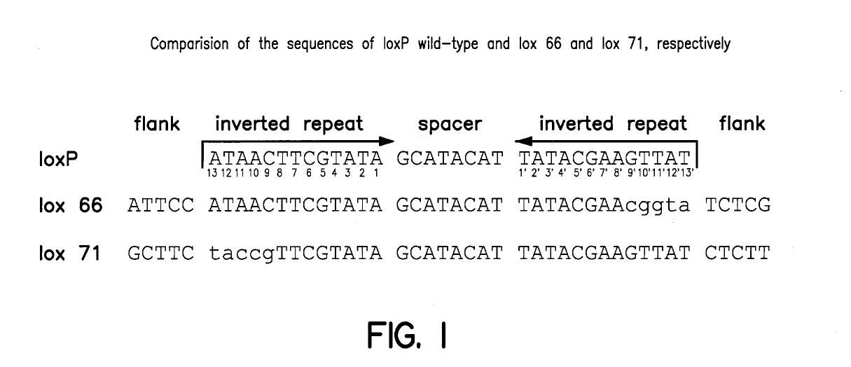

Cloning of the loxP test system is based on pACYC184. The tetracycline resistance gene was excised with HindIII and AvaI followed by insertion of a fragment containing two mutated loxP sequences, lox 66 and lox 71 (FIG. 1). The DNA fragment containing lox 66 and lox 71 was generated after annealing of two partially overlapping oligonucleotides (5′-GGGAAGCTTCTACCGTTCGTATAGCATACATTATACGAAGTTATCTCTTGCGGG ATATCGTCCATTCC-3′ and 5′-CCCCCGAGATACCGTTCGTATAATGTATGCTA-TACGAAGTTATGGAATGGACGATATCCCGCAAGAG-3′) after Klenow enzyme-mediated synthesis of a double stranded DNA fragment and digestion with HindIII and AvaI. This plasmid was called p2627.

In the next step, an Eco47III fragment containing the tetracycline resistance gene of plasmid pACYC184 was inserted into the unique EcoRV recognition sequence between the two mutated loxP sequences of p2627 generating p2632. This plasmid was cut with AseI and partially with PvuII. The fragment harboring the tetracycline resistance gene flanked by the two mutated loxP sites together with the chloramphenicol resistance gene was inserted into pUC19 digested with NdeI and SmaI. The resulting plasmid was designated p2722 and carries three antibiotic resistance genes one of which is flanked by the mutated loxP loci.

E. coli cells carrying p2722 were transfected with a second plasmid, p2676, which replicates via the temperature-sensitive origin of pSC101 (17) and encodes Cre as well as a kanamycin resistance gene. Propagation of the cells in the presence of kanamycin at 30° C. over night results in Cre-mediated removal of the tetracycline resistance gene in the resident plasmid p2722 followed by propagation of tetracycline-sensitive colonies at 42° C. which leads to the loss of p2676. The resulting plasmid p2723 now carries the recombined and mutated loxP locus called lockP which was confirmed by DNA sequencing.

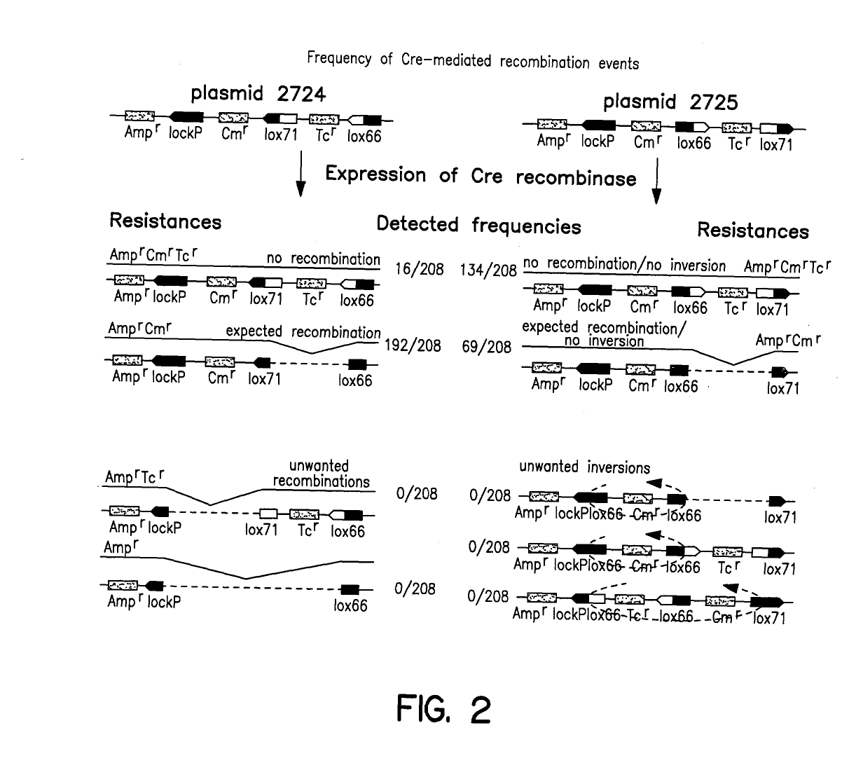

The two final test plasmids were prepared by inserting an AseI/AvaI fragment blunt ended by Klenow enzyme into the BamHI site of p2723 modified in the same manner wherein the fragment is derived from p2632. The p2632 derived AseI/AvaI fragment contains the two mutated loxP sequences lox26 and lox71 flanking the tetracycline resistance gene of pACYC184. The two final plasmids, p2724 and p2725 (FIG. 2), harbor the lox66/lox71 flanked tetracycline resistance gene in both possible orientations with respect to the lockP site and the loci encoding resistance to chloramphenicol and ampicillin (FIG. 2). Both test plasmids, p2724 and p2725 (FIG. 2), carry an identical set of three antibiotic resistance genes but differ with respect to the relative orientation of the three loxP variants lox66, lox71 and lockP. In p²⁷²⁴ all three loxP loci are arranged in the same orientation while in p2725 the lox66 and lox71 loci and the tetracycline resistance gene in-between are inverted relative to the lockP locus with respect to their 8 bp spacer sequences (FIG. 1). E. coli cells harboring either p2724 or p2725 were transformed with an expression plasmid (p2676) encoding Cre which also provides resistance to kanamycin and replicates via a temperature-sensitive origin of DNA replication. One hour following DNA transformation and phenotypic expression at 30° C. (the permissive temperature of p2676), kanamycin and ampicillin were added to the liquid culture and incubation of the cells was continued for 16 hours. Plasma DNA was generated and 10 pg were transfected into E. coli strain DH5α using standard procedures (18). The cells were plated onto LB plates containing ampicillin at 37° C. over night. After incubation over night at 37° C., ampicillin-resistant cells were examined on replica plates containing ampicillin, combinations of ampicillin/tetracycline, ampicillin/chloramphenicol, and ampicillin/tetracycline/chloramphenicol. The number of colonies on the different replica plates in the presence of different antibiotics gave a first indication as to the usefulness of loxP sites recombined by Cre (FIG. 2).

In the case of both test plasmids colonies growing in the presence of all three antibiotics would not have undergone any recombination. Alternatively, these colonies could contain modified test plasmids having inverted DNA segments between the different loxP sites. Colonies growing in the presence of ampicillin and chloramphenicol, but not in the presence of tetracycline presumably have recombined as expected between lox66 and lox71 but not via lockP. The loss of a chloramphenicol resistance or of both the chloramphenicol and the tetracycline resistances would indicate that the lockP locus was involved in Cre-mediated recombination events resulting in the desired recombination (FIG. 2).

Eight colonies obtained from experiments with either test plasmid p2724 or p2725 exhibiting the expected phenotypic pattern (chloramphenicol and ampicillin resistance, tetracycline sensitivity) and 48 colonies from plasmid 2725 which had not undergone any recombination as indicated by their phenotype (resistance to chloramphenicol, ampicillin, and tetracycline) were further examined by means of restriction enzyme analysis. All plasmid DNAs derived from the independent colonies showed the predicted restriction pattern (data not shown) expected from their phenotypes as determined by replica plating. In none of the cases an inversion of DNA segments between the different loxP loci was found. 5 clones derived from test plasmid p2725 showing an unexpected resistance pattern suffered from a complete rearrangement of p2725 which could not be explained by Cre-mediated use of any of the mutated loxP sequences (data not shown). In none of the cases the mutated lockP sequence served as a Cre substrate.

In summary our analysis demonstrates that Cre-mediated recombination between two mutated loxP loci, lox66 and lox71, efficiently occurs if the loxP loci are arranged in a direct orientation as in p2724. It is not apparent why Cre-mediated recombination is less efficient with test plasmid p2725. This plasmid contains the loxP sequences in two orientations which might interfere with the recombination activity of Cre. In all cases it was even more important, however, that the resulting lockP sequence was completely refractory to Cre-mediated recombination indicating the functional inactivation of lockP as a result of a previous site-specific Cre recombination.

This result was surprising for two reasons. First, the same core sites lox 66 and lox 71 (FIG. 1 with different nucleotides flanking the loxP motifs) have been reported to be substrates of Cre although with reduced efficacy as compared to the wild-type loxP. Nevertheless, the lox66 and lox71 loci retained about one fifth of their recombinatory activity after the lockP motif was formed (14). Second, the protein structure of Cre together with biochemical binding experiments has demonstrated that positions 2, 3, 6, and 7 of loxP (FIG. 1) were most important for binding of Cre to loxP whereas the positions beyond 9 did not seem to be of much importance for binding of Cre to its target motif (19, 20).

The two loxP sequences examined in this Example have completely lost their recombination efficacy after site-specific recombination. For this reason multiple consecutive loxP-Cre-mediated recombination events may be carried out within a single cell or even on a single DNA molecule.

### REFERENCES

1. Sternberg, N., Austin, S., Hamilton, D. and Yarmolinsky, M. (1978) Analysis of bacteriophage P1 immunity by using lambda-P1 recombinants constructed in vitro. Proc Natl Acad Sci U S A, 75, 5594-5598.

2. Mack, A., Sauer, B., Abremski, K. and Hoess, R. (1992) Stoichiometry of the Cre recombinase bound to the lox recombining site. Nucleic Acids Res, 20, 4451-4455.

3. Hoess, R., Abremski, K., Irwin, S., Kendall, M. and Mack, A. (1990) DNA specificity of the Cre recombinase resides in the 25 kDa carboxyl domain of the protein. J Mol Biol, 216, 873-882.

4. Dymecki, S. M. (2000) Site-specific recombination in cells and mice. In Joyner, A. L. (ed.), Gene targeting—a practical approach. 2nd Ed. Oxford University Press, Oxford, pp. 37-99.

5. Torres, R. M. and Kühn, R. (1997) Laboratory protocols for conditional gene targeting, Oxford University Press, Oxford.

6. Shizuya, H., Birren, B., Kim, U. J., Mancino, V., Slepak, T., Tachiiri, Y. and Simon, M. (1992) Cloning and stable maintenance of 300-kilobase-pair fragments of human DNA in Escherichia coli using an F-factor-based vector. Proc Natl Acad Sci U S A, 89, 8794-8797.

7. Ioannou, P. A., Amemiya, C. T., Garnes, J., Kroisel, P. M., Shizuya, H., Chen, C., Batzer, M. A. and de Jong, P. J. (1994) A new bacteriophage P1-derived vector for the propagation of large human DNA fragments. Nat Genet, 6, 84-89.

8. Shepherd, N. S., Pfrogner, B. D., Coulby, J. N., Ackerman, S. L., Vaidyanathan, G., Sauer, R. H., Balkenhol, T. C. and Sternberg, N. (1994) Preparation and screening of an arrayed human genomic library generated with the P1 cloning system. Proc Natl Acad Sci U S A, 91, 2629-2633.

9. O'Connor, M., Peifer, M. and Bender, W. (1989) Construction of large DNA segments in Escherichia coli. Science, 244,1307-1312.

10. Kempkes, B., Pich, D., Zeidler, R. and Hammerschmidt, W. (1995) Immortalization of human primary B-lymphocytes in vitro with DNA. Proc Natl Acad Sci U S A, 92, 5875-5879.

11. Brune, W., Messerle, M. and Koszinowski, U. H. (2000) Forward with BACs: new tools for herpesvirus genomics. Trends Genet, 16, 254-259.

12. Muyrers, J. P., Zhang, Y., Testa, G. and Stewart, A. F. (1999) Rapid modification of bacterial artificial chromosomes by ET-recombination. Nucleic Acids Res, 27, 1555-1557.

13. Lee, G. and Saito, I. (1998) Role of nucleotide sequences of loxP spacer region in Cre-mediated recombination. Gene, 216, 55-65.

14. Albert, H., Dale, E. C., Lee, E. and Ow, D. W. (1995) Site-specific integration of DNA into wild-type and mutant lox sites placed in the plant genome. Plant J, 7, 649-659.

15. Sauer, B. (1996) Multiplex Cre/lox recombination permits selective site-specific DNA targeting to both a natural and an engineered site in the yeast genome. Nucleic Acids Res, 24, 4608-4613.

16. Araki, K., Araki, M. and Yamamura, K. (1997) Targeted integration of DNA using mutant lox sites in embryonic stem cells. Nucleic Acids Res, 25, 868-872.

17. Chung, C. T., Niemela, S. L. and Miller, R. H. (1989) One-step preparation of competent Escherichia coli: transformation and storage of bacterial cells in the same solution. Proc Natl Acad Sci U S A, 86, 2172-2175.

18. Hanahan, D. (1985) Techniques for transformation of E. coli. In Glover, D. (ed.), DNA cloning. A practical approach. IRL Press, Oxford, Vol. 11 pp. 109-135.

19. Hartung, M. and Kisters-Woike, B. (1998) Cre mutants with altered DNA binding properties. J Biol Chem, 273, 22884-22891.

20. Guo, F., Gopaul, D. N., and van Duyne, G. D. (1997) Structure of Cre recombinase complexed with DNA in a site-specific recombination synapse. Nature, 389, 40-46.

1. The use of two non-identical recognition sequence mutants for sequence-specific recombinases wherein each of the recognition sequence mutants comprises two recognition sequences separated by a spacer sequence, and wherein each mutant carries mutations in one of the recognition sequences which are inversely repetitive to each other and the respective other recognition sequence corresponds to the wild-type recognition sequence, for performing two or more recombination events by means of a sequence-specific recombinase in a single genetic system.

2. The use according to claim 1 wherein the recognition sequence mutants are employed in the context of the Cre/loxP system.

3. The use according to claim 2 wherein the two loxP mutants lox 66 and lox 71 according to SEQ ID NOS. 1 and 6 are employed.

4. The use according to claims 2 and 3 wherein any of the sequences shown in SEQ ID NOS. 2-5 and 7-10 is employed as the recognition sequence mutant.

5. The use according to claims 3 and 4 wherein the recognition sequence mutants of SEQ ID NOS. 1-5 are flanked in 5′→3′ direction by the sequences ATTCC and TCTCG, and the recognition sequence mutants of SEQ ID NOS. 6-10 are flanked by the sequences GCTTC and CTCTT.

6. The use according to claim 1 wherein the recognition sequence mutants are employed in the context of the Saccharomyces cerevisiae Flp-FRT, Zygosaccharomyces rouxii pSR1, the resolvase-rfsF and the phage Mu Gin recombinase system.

7. A method of recombination for performing multiple recombinations by means of a recombinase in a single genetic system comprising the following steps:
a) Providing two non-identical recognition sequence mutants for sequence-specific recombinases wherein each of the recognition sequence mutants comprises two recognition sequences separated by a spacer sequence, and wherein each mutant carries mutations in one of the recognition sequences which are inversely repetitive to each other and the respective other recognition sequence corresponds to the wild-type recognition sequence, and wherein the two recognition sequence mutants are arranged to have their wild-type sequences on the side facing the site of recombination;
b) induction of a sequence-specific recombinase to carry out a recombination event leaving a recognition sequence mutant which is no longer recognized by the recombinase
c) repeating steps a)+b) to perform further recombination events.

8. A method of recombination according to claim 7 wherein Cre recombinase is employed and wherein the two loxP mutants are lox 66 and lox 71 according to SEQ ID NOS. 1 and 6.

9. A method according to claim 7 wherein Cre recombinase is employed and any of the sequences shown in SEQ ID NOS. 2-5 and 7-10 is employed as the recognition sequence mutant.

10. A method according to claim 8 or 9 wherein the recognition sequence mutants of SEQ ID NOS. 1-5 are flanked in 5′→3′ direction by the sequences ATTCC and TCTCG, and the recognition sequence mutants of SEQ ID NOS. 6-10 are flanked by the sequences GCTTC and CTCTT.

11. A method of recombination according to any of the claims 8-10 wherein Cre recombinase is encoded by a vector expressed in the genetic system.

12. A method according to claim 7 wherein the recognition sequence mutants are employed in the context of the Saccharomyces cerevisiae Flp-FRT, Zygosaccharomyces rouxii pSR1, the resolvase-rfsF and the phage Mu Gin recombinase system.

13. A method of recombination according to any of the claims 7-12 wherein the recombination event is comprised by insertion of a DNA sequence.

14. A method of recombination according to any of the claims 7-12 wherein the recombination event is comprised by excision of a DNA sequence.

15. A method of recombination according to claim 14 wherein the DNA sequence comprises a marker gene.

16. A method of recombination according to claim 15 wherein the marker gene is an antibiotic resistance gene.

17. A method of recombination according to claim 16 wherein the antibiotic resistance gene confers resistance to chloramphenicol, tetracycline, or ampicillin.

18. A method of recombination according to any of the claims 8-11 wherein the loxP mutant generated upon the first recombination event has the same orientation as the loxP mutants provided for performing further recombination events.

19. A recognition sequence mutant characterized by the nucleic acid sequences according to SEQ ID NOS. 2-5 and 7-10.


SEQUENCE LISTING (15 sequences)

SEQ ID NO: 1 - DNA, 34 bp, Artificial sequence
  Description of artificial sequence Oligonucleotide lox 66 without flanks
ataacttcgtatagcatacattatacgaacggta

SEQ ID NO: 2 - DNA, 34 bp, Artificial sequence
  Description of artificial sequence Oligonucleotide
ataacttcgtatagcatacattatacgcacggta

SEQ ID NO: 3 - DNA, 34 bp, Artificial sequence
  Description of artificial sequence Oligonucleotide
ataacttcgtatagcatacattatacgcccggta

SEQ ID NO: 4 - DNA, 34 bp, Artificial sequence
  Description of artificial sequence Oligonucleotide
ataacttcgtatagcatacattataggtaccgta

SEQ ID NO: 5 - DNA, 34 bp, Artificial sequence
  Description of artificial sequence Oligonucleotide
ataacttcgtatagcatacattatacgtaccggg

SEQ ID NO: 6 - DNA, 34 bp, Artificial sequence
  Description of artificial sequence Oligonucleotide lox 71 without flanks
taccgttcgtatagcatacattatacgaagttat

SEQ ID NO: 7 - DNA, 34 bp, Artificial sequence
  Description of artificial sequence Oligonucleotide
tagcgttcgtatagcatacattatacgaagttat

SEQ ID NO: 8 - DNA, 34 bp, Artificial sequence; identical to SEQ ID NO: 6
  Description of artificial sequence Oligonucleotide

SEQ ID NO: 9 - DNA, 34 bp, Artificial sequence
  Description of artificial sequence Oligonucleotide
taccgggcgtatagcatacattatacgaagttat

SEQ ID NO: 10 - DNA, 34 bp, Artificial sequence
  Description of artificial sequence Oligonucleotide
aatgcatgctatagcatacattatacgaagttat

SEQ ID NO: 11 - DNA, 68 bp, Artificial sequence
  Description of artificial sequence Oligonucleotide
gggaagcttctaccgttcgtatagcatacattatacgaagttatctcttgcgggatatcgtccattcc

SEQ ID NO: 12 - DNA, 67 bp, Artificial sequence
  Description of artificial sequence Oligonucleotide
cccccgagataccgttcgtataatgtatgctatacgaagttatggaatggacgatatcccgcaagag

SEQ ID NO: 13 - DNA, 34 bp, Artificial sequence
  Description of artificial sequence Oligonucleotide loxP
ataacttcgtatagcatacattatacgaagttat

SEQ ID NO: 14 - DNA, 44 bp, Artificial sequence
  Description of artificial sequence Oligonucleotide lox 66 with flanks
attccataacttcgtatagcatacattatacgaacggtatctcg

SEQ ID NO: 15 - DNA, 44 bp, Artificial sequence
  Description of artificial sequence Oligonucleotide lox 71 with flanks
gcttctaccgttcgtatagcatacattatacgaagttatctctt